(19) United States
(12) Patent Application Publication
(10) Pub. No.: US 2022/0409858 A1
(43) Pub. Date: Dec. 29, 2022

(54) ELECTROMAGNETIC-RADIATION-CURED RADIOPAQUE MARKER AND ASSOCIATED DEVICES, SYSTEMS, AND METHODS

(71) Applicants: PHILIPS IMAGE GUIDED THERAPY CORPORATION, SAN DIEGO, CA (US); KONINKLIJKE PHILIPS N.V., Eindhoven (NL)

(72) Inventor: Tung-Liang LIN, ESCONDIDO, CA (US)

(21) Appl. No.: 17/777,168

(22) PCT Filed: Nov. 27, 2020
(86) PCT No.: PCT/EP2020/083628

§ 371 Date: May 16, 2022

Related U.S. Application Data
(60) Provisional application No. 62/940,369, filed on Nov. 26, 2019.

(51) Int. Cl. A61M 25/01; A61M 25/09; A61B 8/12

(52) U.S. Cl. CPC A61M 25/0108; A61M 25/09; A61B 8/12; A61M 2025/09166; A61B 2090/3966

(57) ABSTRACT

An intraluminal sensing device includes a catheter or a guidewire with a flexible elongate member that is positioned within a body lumen of a patient. The device also includes a sensor disposed at a distal portion of the flexible elongate member. The sensor obtains medical data associated with the body lumen while the flexible elongate member is positioned within the body lumen. The sensor is an ultrasound transducer, a pressure sensor, a flow sensor, and/or a temperature sensor. The device further includes a radiopaque marker coupled to the flexible elongate member. The radiopaque marker is an ultraviolet (UV) radiation-cured product of a mixture that includes a radiopaque material, an electromagnetic-radiation-curable adhesive, and a photoinitiator. The solution of the mixture is applied directly to the flexible elongate member and UV radiation-cured to form the radiopaque marker. The radiopaque marker is a band extending around a perimeter of the flexible elongate member.

### FIELD OF DISCLOSURE

The present disclosure relates to a medical device with a radiopaque marker formed of a mixture of a radiopaque material, an adhesive, and a photoinitiator that is electromagnetic-radiation-cured. Exemplary medical devices with such a radiopaque marker include a catheter, a guide wire, a guide catheter, a gastroscope, and/or other intraluminal devices.

### BACKGROUND

A medical device can be visualized within the body of a patient using x-ray imaging by providing a radiopaque marker on the medical device. Thus, even if the medical device is predominately formed of a radiolucent material, a clinician can still see a radiopaque portion of the medical device in the x-ray image. Some existing radiopaque markers are formed of a radiopaque metal that is attached to a medical device. Metal radiopaque marker bands, however, are relatively rigid (in applications for which a flexible medical device is needed), more expensive, and can become dislodged. Some existing radiopaque markers are polymer-based. Polymer marker band tubing is produced by extruding a blend of polymer resin (a solid) and radiopaque material. After extrusion, the polymer band tube is cut into a pre-defined length and attached to a device using thermal or laser bonding. Existing polymer radiopaque markers thus require extrusion, cutting, and thermal/laser bonding. These steps add time to the manufacturing process, as well as expense for the machines to perform extrusion, cutting, and thermal/laser bonding.

### SUMMARY

The present disclosure describes a radiopaque marker on a medical device that is visible in an x-ray image. The radiopaque marker is cured mixture of adhesive(s) (e.g., an acrylated urethane, acrylate, oligomer, and/or monomer), radiopaque material(s), and photoinitiator(s). The mixture is a liquid that is applied onto the surface of the medical device and cured using electromagnetic radiation (such as ultraviolet radiation). During curing, the liquid solution polymerizes into the radiopaque marker. The radiopaque marker band can be flexible and advantageously implemented on a flexible medical device, such as a catheter, guide wire, or guide catheter, which traverse tortuous vasculature within a patient's body. In that regard, the radiopaque marker can be formed while avoiding the time and expense associated with extrusion, cutting, and thermal/laser bonding.

In an embodiment of the present disclosure, an intraluminal device is provided. The device includes a catheter or a guidewire comprising a flexible elongate member configured to be positioned within a body lumen of a patient; and a radiopaque marker coupled to the flexible elongate member, wherein the radiopaque marker is an electromagnetic-radiation-cured product of a mixture comprising: a radiopaque material; an electromagnetic-radiation-curable adhesive; and a photoinitiator.

In some embodiments, the electromagnetic-radiation-curable adhesive comprises at least one of an acrylated urethane or an acrylate. In some embodiments, the electromagnetic-radiation-curable adhesive comprises at least one of a monomer or an oligomer. In some embodiments, the radiopaque marker is an ultraviolet (UV)-radiation-cured product. In some embodiments, the mixture comprises a plurality of electromagnetic-radiation-curable adhesives. In some embodiments, the flexible elongate member comprises a surface, and the radiopaque marker conforms to the surface. In some embodiments, the radiopaque marker comprises a band extending around a perimeter of the flexible elongate member. In some embodiments, the device further includes a sensor disposed at a distal portion of the flexible elongate member and configured to obtain medical data associated with the body lumen while the flexible elongate member is positioned within the body lumen. In some embodiments, the sensor comprises at least one of an ultrasound transducer, a pressure sensor, a flow sensor, or a temperature sensor. In some embodiments, the radiopaque material is between approximately 30% and approximately 90% of a weight of the mixture.

In an embodiment of the present disclosure, a medical device is provided. The devices includes a surface; and a radiopaque marker coupled to the surface, wherein the radiopaque marker is an electromagnetic-radiation-cured product of a mixture comprising: a radiopaque material; an electromagnetic-radiation-curable adhesive; and a photoinitiator.

In an embodiment of the present disclosure, an intraluminal sensing device is provided. The device includes a catheter or a guidewire comprising a flexible elongate member configured to be positioned within a body lumen of a patient; a sensor disposed at a distal portion of the flexible elongate member and configured to obtain medical data associated with the body lumen while the flexible elongate member is positioned within the body lumen, wherein the sensor comprises at least one of an ultrasound transducer, a pressure sensor, a flow sensor, or a temperature sensor; and a radiopaque marker coupled to the flexible elongate member, wherein the radiopaque marker is an ultraviolet (UV) radiation-cured product of a mixture comprising: a radiopaque material; an electromagnetic-radiation-curable adhesive; and a photoinitiator, wherein a solution of the mixture is applied directly to the flexible elongate member and UV radiation-cured to form the radiopaque marker, wherein the radiopaque marker comprises a band extending around a perimeter of the flexible elongate member.

Additional aspects, features, and advantages of the present disclosure will become apparent from the following detailed description.

### BRIEF DESCRIPTION OF THE DRAWINGS

The following descriptions of the drawings are merely an embodiment of the disclosure and should not be considered limited. Also, the drawings are merely a depiction of embodiments and are not drawn to scale.

FIG. 1 is a diagrammatic side view of a radiopaque marker coupled to a medical device, according to embodiments of the present disclosure.

FIG. 2 is flow diagram of a method of manufacturing a medical device with a radiopaque marker, according to embodiments of the present disclosure.

FIG. 3 is a diagrammatic cross-sectional end view of a radiopaque marker coupled to a guide wire, according to embodiments of the present disclosure.

FIG. 4 is a diagrammatic cross-sectional end view of a radiopaque marker coupled to an inner member of a catheter, according to embodiments of the present disclosure.

FIG. 5 is a diagrammatic side view of a sensing guide wire, according to embodiments of the present disclosure.

FIG. 6 is a diagrammatic side view of a distal portion of the sensing guide wire of FIG. 5, according to embodiments of the present disclosure.

FIG. 7 is a diagrammatic view of an intravascular ultrasound (IVUS) imaging system, according to embodiments of the present disclosure.

FIG. 8 is a diagrammatic view of an imaging system, according to embodiments of the present disclosure.

FIG. 9 is a diagrammatic partial cutaway perspective view of an imaging device, according to embodiments of the present disclosure.

FIG. 10 is a diagrammatic cross-sectional side view of a distal portion of an imaging device, according to embodiments of the present disclosure.

### DETAILED DESCRIPTION

The term “about” indicates a range which includes ±5% when used to describe a single number. When applied to a range, the term “about” indicates that the range includes −5% of a numerical lower boundary and +5% of an upper numerical boundary. For example, a range of from about 100° C. to about 200° C., includes a range of from 95° C. to 210° C. A range of about 5:1 to about 1:5 includes a range of from 5.25: 1 to 0.95:5. However, when the term “about” modifies a percentage, then the term means ±1% of the number or numerical boundaries, unless the lower boundary is 0%. Thus, a range of 5-10%, includes 4-11%. A range of 0-5%, includes 0-6%.

Unless indicated otherwise, the terms “a,” “an,” or “the” can refer to one or more than one of the noun they modify.

The phrase “section of a medical device” refers to a length of a medical device that will be exposed to the environment during intravascular medical procedures. For example, the environment can be within a patient's body or outside of the patient's body.

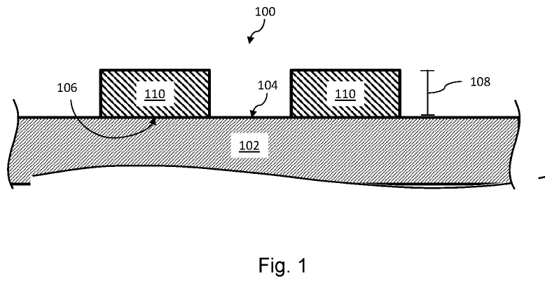

FIG. 1 is a diagrammatic side view of a medical device 100 including a radiopaque marker 110, according to an embodiment of the present disclosure. In some embodiments, FIG. 1 is a cross-sectional view. The radiopaque marker 110 is coupled to a body 102 of the medical device 100. In some embodiments, the radiopaque marker 110 is formed of a liquid solution that is applied to a surface 106 of the body 102 and cured such that the radiopaque marker 110 polymerizes on the surface 106. Accordingly, in such embodiments, the radiopaque marker 110 is not a piece of metal that is coupled to the body 102 via adhesive. Additionally, the radiopaque marker 110 is not a cut length of an extruded blend of polymer resin and radiopaque material that is bonded to the body 102 via thermal and/or laser bonding. The radiopaque marker 110 is visible in an x-ray image, such as an angiographic or fluoroscopic image.

The radiopaque marker 110 can be referenced as a polymer/polymeric/polymerized radiopaque marker, an adhesive-based radiopaque marker 110, or a flexible radiopaque marker 110, for example. The radiopaque marker 110 can be an electromagnetic-radiation-cured product of a mixture including a radiopaque material, an electromagnetic-radiation-curable adhesive, and a photoinitiator. The mixture can be a liquid solution. The viscosity of the solution can vary based on the particular components of the mixture. The liquid solution can be cured using any suitable wavelength of electromagnetic radiation. For example, the radiopaque marker 110 can be ultraviolet (UV) radiation cured using, e.g., UVA, UVB, and/or UVC. The radiopaque marker 110 can also be cured using visible light. For example, the radiopaque marker 110 can be cured using light from a light emitting diode (LED) or other suitable electromagnetic radiation source. During curing, the liquid solution polymerizes to form the radiopaque marker 110. For example, the mixture can solidify and/or harden, relative to the liquid solution. The radiopaque marker 110 is a cured polymer after curing. In some embodiments, such as those in which the body 102 of the medical device 100 is flexible, the radiopaque marker 110 cures into a flexible product such that the portion of the body 102 with the radiopaque marker 110 remains flexible.

The radiopaque material in the mixture can be a metal, metal alloy, and/or an inorganic compound. In some embodiments, the radiopaque material is a heavy metal. In some embodiments, the radiopaque material is a solid, such as a powder of any suitable particle size (e.g., about 1 micron to about 5 microns). Any suitable radiopaque material, such as tungsten, barium sulfate, bismuth trioxide, bismuth subcarbonate, or bismuth oxychrolide, can be used. In some embodiments, the mixture includes one or multiple radiopaque materials. Varying amounts of radiopaque material can be included in the mixture. In some embodiments, the radiopaque material can be between about 30% and about 90% of the mixture by weight.

The adhesive in the mixture can be any suitable electromagnetic-radiation-curable adhesive. In some embodiments, the adhesive can be a monomer and/or an oligomer. For example, the adhesive could be monofunctional monomers, such as isobornyl acrylate and N,N-dimethylacrylamide. In some embodiments, the adhesive can be an acrylated urethane oligomer and/or a di-functional or tri-functional acrylate. For example, acrylated urethane oligomer adhesives could be Photomer 6210, Photomer 6230, Photomer 6891, Photomer 6892, available from IGM Resins, and polybutadiene dimethacrylate available from Sartomer. For example, di-functional or tri-functional acrylate could be trimethylolpropane triacrylate (TMPTA) and/or polyethylene glycol dimethacrylate available from Sigma-Aldrich. In some embodiments, the mixture includes one or more adhesion promoters, such as Photomer 4173 and Photomer 4703 available from IGM Resins, to promote adhesion of the marker 100 to the medical device 100. The adhesive is in a liquid form. The viscosity of the liquid will vary based on the particular adhesive. The mixture can include a monomer and an oligomer. The mixture can include an acrylate and an acrylated urethane oligomer. In some embodiments, multiple adhesives can be included in the mixture. For example, the mixture can include multiple monomers and/or multiple oligomers. The mixture can include multiple urethanes and/or multiple acrylates. In various embodiments, one, two, three, four, five, or more adhesives can be included in the mixture.

The one or more adhesives in the mixture can be advantageously selected based on the desired properties of the mixture solution and/or the cured radiopaque marker 110. For example, the one or more adhesives can be advantageously selected to have the desired viscosity to ensure effective application of the liquid solution to the medical device. For example, the liquid solution can be sufficiently viscous that it remains on (and does not slide off of) the medical device and not so viscous that it is difficult to dispense the liquid solution onto the medical device. For example, the viscosity can be suitable for dispensing, spraying, and/or printing the solution onto the medical device. The one or more adhesives can also be advantageously selected to have the desired flexibility for the radiopaque marker 110 such that, e.g., a flexible radiopaque marker 110 can be provided for the flexible body 102 of the medical device 100. In some embodiments, the adhesive(s) can be selected to good adhesion strength to the substrate to which the radiopaque maker 110 is coupled (e.g., the body 102 of the medical device 100).

The polymerization initiator in the mixture can be any suitable photoinitiator. In some embodiments, the initiator is a free radical initiator. In the presence of the electromagnetic radiation during curing, the photoinitiator breaks down, releases reactive radicals, and initiates polymerization of the liquid solution. The broken down photoinitiator will become part of cured polymer in the final product of the radiopaque marker 110. The photoinitiator can be advantageously selected based on the electromagnetic radiation that will be used to cure the mixture. For example, a suitable photoinitiator can be selected based on whether UVA, UVB, or UVC is used for curing. Example photoinitiators include Omnirad TPO, Omnirad 2100, and/or Omnirad 2022 available from IGM Resins. The photoinitiator can be a liquid or a solid. In some embodiments, multiple photoinitiators can be included in the mixture.

Referring still to FIG. 1, the radiopaque marker 110 can have any suitable dimensions, such as length, width, height, thickness, perimeter, circumference, etc. For example, the height or thickness 108 of the radiopaque marker 110 can be between about 30 microns and 100 microns, in some embodiments.

The medical device 100 can include one or multiple radiopaque markers 110. FIG. 1 illustrates two radiopaque markers 110. The radiopaque markers 110 are spaced from one another. The radiopaque markers 110 can be spaced from one another by a fixed, known distance such that the radiopaque markers 110 can be used to measure a length of the anatomy using the x-ray image. Any suitable quantity of radiopaque markers 110 can be provided, including one, two, three, four, five, or more.

A surface 106 of the one or more radiopaque markers 110 is coupled to surface 104 of the body 102 of the medical device 100. The surface 106 can be an inner surface of the radiopaque marker 110. The surface 104 can be an outer surface or an inner surface of the body 102. The radiopaque marker 110 is in direct contact with the body 102 at the surfaces 104, 106. The surface 106 of the radiopaque marker 110 can conform to and have the same shape as the surface 104 of the body 102. The radiopaque marker 110 can be disposed on all or a portion of the entire surface 104 of the body 102. For example, one or more radiopaque markers 110 can be selectively disposed on the medical device to provide visual guidance in an x-ray image about the location of the medical device 100 within the patient body. In some embodiments, the radiopaque marker 110 can cover between about 1% and about 100% of one or more surfaces of one or more sections of the medical device 100. The radiopaque marker 110 can have any suitable two-dimensional or three-dimensional shape. For example, the radiopaque marker 110 can be polygon, polyhedron, ellipse, ellipsoid, and/or combinations thereof. The radiopaque marker 110 can be a compound shape formed of multiple individual shapes. In some embodiments, the radiopaque marker 110 can be a band, annulus, and/or ring that extends around a perimeter of the body 102 of the medical device 100. In some embodiments, the radiopaque component 110 is not necessarily coupled to the body 102 as a separate component, and rather itself forms all or a portion of the body 102 of the medical device 100. While the radiopaque marker is described herein, it is understood that there are other uses for the electromagnetic radiation curable component 110.

The body 102 of the medical device 100 may be rigid or flexible. The body 102 can be formed of any suitable material, such plastic, polymer, metal, metal alloy, and/or combinations thereof. The medical device 100 can be any suitable diagnostic and/or therapeutic device. In some embodiments, the medical device 100 can be an intraluminal device that is configured to positioned within a body lumen of a patient. In that regarding, the medical device 100 can be temporarily positioned within the body lumen (e.g., during a medical procedure) or disposed within the body for an extended period of time (e.g., a stent or a pacemaker). In some embodiments, the medical device 100 can be a guidewire, a catheter, or a guide catheter. In some embodiments, the medical device 100 can be a probe, such as a gastroscope, an endoprobe, a transvaginal probe, a transrectal probe, and/or other intrabody probe. The body 102 can be rigid or flexible. For example, the body 102 can be a flexible elongate member. For example, a distal portion of the flexible elongate member can be positioned within the patient body while a proximal portion remains outside of the patient body. The body 102 can be a shaft of the medical device 100 in some embodiments. The medical device 100 and/or the body 102 can be sized and shaped, structurally arranged and/or otherwise configured for insertion into a vessel or lumen of the patient.

The medical device 100 can include a sensor. The sensor can be coupled to and/or otherwise disposed at a distal portion of the body 102. The sensor can be configured to obtain medical data associated with the patient's body. For example, the sensor can be an intraluminal sensor configured to obtain medical data while positioned within the patient's body.

The sensor can be an imaging sensor. For example, the imaging sensor can be an acoustic and/or ultrasound imaging element and/or an optical imaging element. The imaging sensor can be used for intravascular ultrasound (IVUS) imaging, forward looking intraluminal ultrasound (FL-IVUS) imaging, intraluminal photoacoustic (IVPA) imaging, intracardiac echocardiography (ICE), forward looking ICE (FLICE), transesophageal echocardiography (TEE), optical coherence tomography (OCT), optical/photographic imaging, and/or other suitable imaging modalities. The medical device 100 can include an ultrasound transducer element, an acoustic element, an array of transducer/acoustic elements, an optical fiber, a reflector, a mirror, and/or a prism.

In some embodiments, the sensor is a non-imaging component, including a pressure sensor, a flow sensor, or a temperature sensor. In some embodiments, the medical device devices include a therapeutic and/or a diagnostic component. For example, a therapeutic tool can include an ablation element, a radio frequency (RF) electrode, an atherectomy blade, atherectomy laser/optical fiber, a balloon, a stent, and/or other suitable component.

Generally, the medical device 100 and/or body 102 can be utilized within any suitable anatomy and/or body lumen of the patient. Lumen may represent fluid filled or fluid-surrounded structures, both natural and man-made. Lumen may be within a body of a patient. Lumen may be a blood vessel, such as an artery or a vein of a patient's vascular system, including cardiac vasculature, peripheral vasculature, neural vasculature, renal vasculature, and/or or any other suitable lumen inside the body. For example, the device may be used to examine any number of anatomical locations and tissue types, including without limitation, organs including the liver, heart, kidneys, gall bladder, pancreas, lungs; ducts; intestines; nervous system structures including the brain, dural sac, spinal cord and peripheral nerves; the urinary tract; as well as valves within the blood, chambers or other parts of the heart, and/or other systems of the body. In addition to natural structures, the medical device 100 and/or body 102 may be used to examine man-made structures such as, but without limitation, heart valves, stents, shunts, filters and other devices.

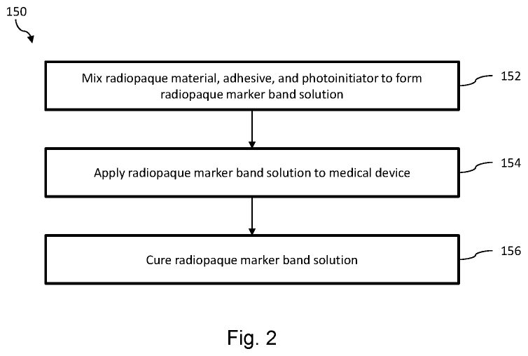

FIG. 2 is flow diagram of a method 150 of manufacturing a medical device with a radiopaque marker, according to embodiments of the present disclosure. As illustrated, the method 150 includes a number of enumerated steps, but embodiments of the method 150 may include additional steps before, after, and in between the enumerated steps. In some embodiments, one or more of the enumerated steps may be omitted, performed in a different order, or performed concurrently. The steps of the method 150 can be carried by a manufacturer to yield the medical devices including features described in FIGS. 1 and 2-10.

At step 152, the method 150 includes mixing a radiopaque material, an adhesive, and a photoinitiator to form a liquid radiopaque marker band solution. In some embodiments, step 152 can include multiple substeps. For example, the adhesive can be mixed with the photoinitiator in one substep. In another substep, the radiopaque material can be added to and mixed with the adhesive/photoinitiator mixture. In some embodiments, the adhesive and photoinitiator can be pre-mixed prior to mixing with the radiopaque material. In some embodiments, the radiopaque material, the adhesive, and the photoinitiator are mixed together at the same time. The radiopaque marker band solution can include one or more radiopaque materials, one or more adhesives, and one or more photoinitiators.

At step 154, the method 150 includes applying a radiopaque marker band solution to the medical device. Any suitable techniques for providing the solution onto one or multiple surfaces of the medical device are contemplated. The solution is applied such that it coats at least a portion of the medical device. For example, the solution can be applied manually or using a machine in automated manner and/or under user control. The solution can be painted, sprayed, printed, and/or otherwise dispensed onto the medical device. In some embodiments, Aerosol jet printing is used to apply the solution to the medical device. In some embodiments, a pre-determined, controlled amount of the solution is dispensed onto the medical device. The solution can be applied at one or multiple locations of the medical device. For example, solution can be dispensed around all or a portion of a perimeter of the medical device. In another example, the solution can be dispensed at one or multiple locations of the medical device and allowed to flow to one or multiple other locations of the medical device.

At step 156, the method 150 includes curing the radiopaque marker band solution. For example, electromagnetic radiation (e.g., UV radiation and or visible light) can be directed to the one or more portions of the medical device with the applied solution. The solution can be exposed to electromagnetic radiation for any suitable time period, depending on the wavelength/energy of the electromagnetic radiation and/or the components of the solution (e.g., the photoinitiator and/or the adhesive). For example, the time period for curing can be between about 10 seconds and 120 seconds. Any suitable techniques for directing electromagnetic radiation onto the radiopaque marker band solution and the medical device is contemplated. A machine, such as a curing lamp, can be used. An example UV curing lamp is the Omnicure S2000 available from Excelitas. The curing lamp can be a mercury lamp.

An example of the method 150 includes preparing, prior to use, a mixture of Photomer 6210, Photomer 4173, TMPTA, polyethylene glycol dimethacrylate, and Ominrad 2100. A UV curable radiopaque marker band solution containing 85% tungsten powder by weight is prepared by mixing 1.5 grams of the adhesive/photoinitiator mixture with 8.5 grams of tungsten powder (2-4 micron particle size). The solution is applied onto the medical device. The solution is cured with UVA radiation using the Omnicure S2000 for 15 seconds.

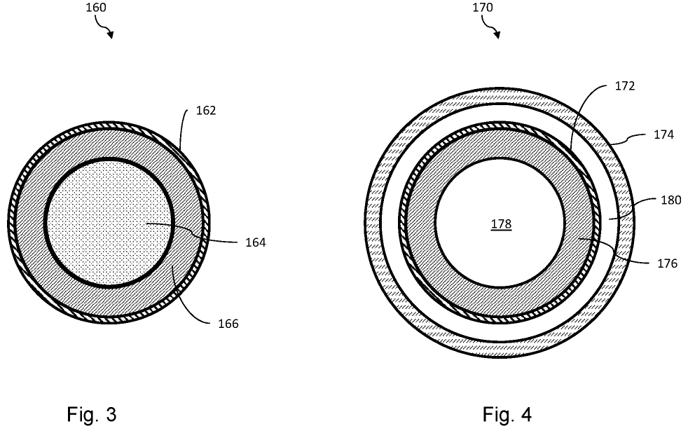

FIG. 3 is a diagrammatic cross-sectional end view of a radiopaque marker 162 coupled to a guide wire 160, according to embodiments of the present disclosure. The guide wire 160 can include a core wire 164 and a polymer layer 166 disposed around the core wire 164. The core wire 164 and/or the polymer layer 166 can form the flexible elongate member of the guide wire 160. In some embodiments, communication lines, such as electrical conductors, extend between the core wire 164 and the polymer layer 166 to provide communication between a sensor at a distal portion of the guide wire 160 and a proximal portion. The core wire 164 is a metallic component that provides structure for the guide wire 160. The radiopaque marker 162 is coupled to an outer surface of the polymer layer 166. In some embodiments, the radiopaque marker 162 is an outermost layer of the guide wire 160. In some embodiments, the radiopaque marker 162 is not the outermost layer, and one or more additional layers (e.g., a hydrophilic coating or a hydrophobic coating) can be provided around and directly in contact with the radiopaque maker 162. An inner surface and/or an outer surface of the radiopaque marker 162 can be directly in contact with another component of the guide wire 160.

FIG. 4 is a diagrammatic cross-sectional end view of a radiopaque marker 172 coupled to an inner member 176 of a catheter 170, according to embodiments of the present disclosure. The catheter 170 can include the inner member 176 and an outer member 174 positioned around the inner member 176. The inner member 176 and/or the outer member 174 can form the flexible elongate member of the catheter 170. The inner member 176 can define a lumen 178 configured to receive a guide wire. The outer member 174 defines an annular lumen 180 around the inner member 176 and/or the radiopaque marker 172. One or more communication lines, such as electrical conductors, can extend within the lumen 180 to provide signal communication with a sensor at the distal portion of the catheter 170 and a proximal portion. All or a portion of the radiopaque marker 172 can be spaced from the outer member 174. In the illustrated embodiment, the radiopaque marker 162 is coupled to and in direct contact with an outer surface of the inner member 176. In other embodiments, the radiopaque marker 162 is coupled to an inner surface of the inner member 176, an inner surface of the outer member 174, and/or an outer surface of the outer member 174.

In the illustrated embodiments of FIG. 3 and FIG. 4, the radiopaque marker 162 extends completely around a perimeter associated with the guidewire 160 and the catheter 170, respectively. In other embodiments, the radiopaque marker 162 extends only partially around the perimeter (e.g., 25%, 33%, 50%, 66%, or 75% around the perimeter).

In several exemplary embodiments, the medical device is not particularly limited so long as it is a medical device that would benefit from having a radiopaque marker for visualization of the medical device in an x-ray image. In several exemplary embodiments, the medical device includes a catheter, such as intravascular ultrasound (IVUS) imaging catheters or microcatheters; a guide wire; a delivery system; a stent, such as a ureteral stent; and the like. “Delivery system” means a delivery catheter or system which is used to deliver devices including a stent, a heart valve, or any implants.

In several exemplary embodiments, the medical device is a sensing guide wire that includes a flexible elongate member; a flexible element extending distally from the flexible elongate member; a core member extending within a lumen of the flexible element; and a sensing element positioned distal of the flexible element.

In several exemplary embodiments, the medical device is a sensing guide wire that includes a flexible elongate member; a variable pitch coil extending distally from the flexible elongate member; and a sensing element coupled to the flexible elongate member.

As used herein, “flexible elongate member” or “elongate flexible member” includes at least any thin, long, flexible structure that can be inserted into the vasculature of a patient. While the illustrated embodiments of the “flexible elongate members” of the present disclosure have a cylindrical profile with a circular cross-sectional profile that defines an outer diameter of the flexible elongate member, in other instances all or a portion of the flexible elongate members may have other geometric cross-sectional profiles (e.g., oval, rectangular, square, elliptical, etc.) or non-geometric cross-sectional profiles. Flexible elongate members include, for example, guide wires and catheters. In that regard, catheters may or may not include a lumen extending along its length for receiving and/or guiding other instruments. If the catheter includes a lumen, the lumen may be centered or offset with respect to the cross-sectional profile of the device.

In most embodiments, the flexible elongate members of the present disclosure include one or more electronic, optical, or electro-optical components. For example, without limitation, a flexible elongate member may include one or more of the following types of components: a pressure sensor, a flow sensor, a temperature sensor, an imaging element, an optical fiber, an ultrasound transducer, a reflector, a minor, a prism, an ablation element, an RF electrode, a conductor, and/or combinations thereof. Generally, these components are configured to obtain data related to a vessel or other portion of the anatomy in which the flexible elongate member is disposed. Often the components are also configured to communicate the data to an external device for processing and/or display. In some aspects, embodiments of the present disclosure include imaging devices for imaging within the lumen of a vessel, including both medical and non-medical applications. However, some embodiments of the present disclosure are particularly suited for use in the context of human vasculature. Imaging of the intravascular space, particularly the interior walls of human vasculature can be accomplished by a number of different techniques, including ultrasound (often referred to as intravascular ultrasound (“IVUS”) and intracardiac echocardiography (“ICE”)) and optical coherence tomography (“OCT”). In other instances, infrared, thermal, or other imaging modalities are utilized.

The electronic, optical, and/or electro-optical components of the present disclosure are often disposed within a distal portion of the flexible elongate member. As used herein, “distal portion” of the flexible elongate member includes any portion of the flexible elongate member from the mid-point to the distal tip. As flexible elongate members can be solid, some embodiments of the present disclosure will include a housing portion at the distal portion for receiving the electronic components. Such housing portions can be tubular structures attached to the distal portion of the elongate member. Some flexible elongate members are tubular and have one or more lumens in which the electronic components can be positioned within the distal portion.

The electronic, optical, and/or electro-optical components and the associated communication lines are sized and shaped to allow for the diameter of the flexible elongate member to be very small. For example, the outside diameter of the elongate member, such as a guide wire or catheter, containing one or more electronic, optical, and/or electro-optical components as described herein are between about 0.0007″ (0.0178 mm) and about 0.118″ (3.0 mm), with some particular embodiments having outer diameters of approximately 0.014″ (0.3556 mm), approximately 0.018″ (0.4572 mm), and approximately 0.035″ (0.889 mm). As such, the flexible elongate members incorporating the electronic, optical, and/or electro-optical component(s) of the present application are suitable for use in a wide variety of lumens within a human patient besides those that are part or immediately surround the heart, including veins and arteries of the extremities, renal arteries, blood vessels in and around the brain, and other lumens.

“Connected” and variations thereof as used herein includes direct connections, such as being glued or otherwise fastened directly to, on, within, etc. another element, as well as indirect connections where one or more elements are disposed between the connected elements.

“Secured” and variations thereof as used herein includes methods by which an element is directly secured to another element, such as being glued or otherwise fastened directly to, on, within, etc. another element, as well as indirect techniques of securing two elements together where one or more elements are disposed between the secured elements.

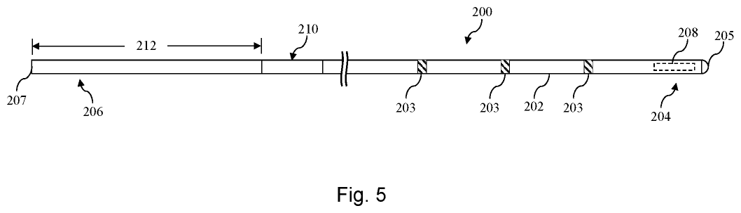

Referring now to FIG. 5, shown therein is an intravascular device 200 according to an embodiment of the present disclosure. In that regard, the intravascular device 200 includes a flexible elongate member 202 having a distal portion 204 adjacent a distal tip 205 and a proximal portion 206 adjacent a proximal end 207. Multiple radiopaque markers 203 are provided along a length of the flexible elongate member 202. The illustrated positions of the radiopaque markers 203 are exemplary. The radiopaque markers 203 can be disposed at the distal portion 204, the proximal portion, and/or an intermediate portion between the distal portion 204 and the proximal portion 206. The radiopaque markers 203 can be positioned along a portion, portions, and/or all of the intravascular device 200.

A component 208 is positioned within the distal portion 204 of the flexible elongate member 202 proximal of the distal tip 205. Generally, the component 208 is representative of one or more electronic, optical, or electro-optical components. In that regard, the component 208 is a pressure sensor, a flow sensor, a temperature sensor, an imaging element, an optical fiber, an ultrasound transducer, a reflector, a minor, a prism, an ablation element, an RF electrode, a conductor, and/or combinations thereof. The specific type of component or combination of components can be selected based on an intended use of the intravascular device. In some instances, the component 208 is positioned less than 10 cm, less than 5, or less than 3 cm from the distal tip 205. In some instances, the component 108 is positioned within a housing of the flexible elongate member 202. In that regard, the housing is a separate component secured to the flexible elongate member 202 in some instances. In other instances, the housing is integrally formed as a part of the flexible elongate member 202.

The intravascular device 200 also includes a connector 210 adjacent the proximal portion 206 of the device. In that regard, the connector 210 is spaced from the proximal end 207 of the flexible elongate member 202 by a distance 212. Generally, the distance 212 is between 0% and 50% of the total length of the flexible elongate member 202. While the total length of the flexible elongate member can be any length, in some embodiments the total length is between about 1300 mm and about 4000 mm, with some specific embodiments have a length of 1400 mm, 1900 mm, and 3000 mm. Accordingly, in some instances the connector 210 is positioned at the proximal end 207. In other instances, the connector 210 is spaced from the proximal end 207. For example, in some instances the connector 210 is spaced from the proximal end 207 between about 0 mm and about 1400 mm. In some specific embodiments, the connector 210 is spaced from the proximal end by a distance of 0 mm, 300 mm, and 1400 mm.

The connector 210 is configured to facilitate communication between the intravascular device 200 and another device. More specifically, in some embodiments the connector 210 is configured to facilitate communication of data obtained by the component 208 to another device, such as a computing device or processor. Accordingly, in some embodiments the connector 210 is an electrical connector. In such instances, the connector 210 provides an electrical connection to one or more electrical conductors that extend along the length of the flexible elongate member 202 and are electrically coupled to the component 208. In some embodiments the electrical conductors are embedded within a core of the flexible elongate member. In other embodiments, the connector 210 is an optical connector. In such instances, the connector 210 provides an optical connection to one or more optical communication pathways (e.g., fiber optic cable) that extend along the length of the flexible elongate member 202 and are optically coupled to the component 208. Similarly, in some embodiments the optical fibers are embedded within a core of the flexible elongate member. Further, in some embodiments the connector 210 provides both electrical and optical connections to both electrical conductor(s) and optical communication pathway(s) coupled to the component 208. In that regard, it should be noted that component 208 is comprised of a plurality of elements in some instances. The connector 210 is configured to provide a physical connection to another device, either directly or indirectly. In some instances, the connector 210 is configured to facilitate wireless communication between the intravascular device 200 and another device. Generally, any current or future developed wireless protocol(s) may be utilized. In yet other instances, the connector 210 facilitates both physical and wireless connection to another device.

As noted above, in some instances the connector 210 provides a connection between the component 208 of the intravascular device 200 and an external device. Accordingly, in some embodiments one or more electrical conductors, one or more optical pathways, and/or combinations thereof extend along the length of the flexible elongate member 202 between the connector 210 and the component 208 to facilitate communication between the connector 210 and the component 208. In some instances, at least one of the electrical conductors and/or optical pathways is embedded within the core of the flexible elongate member 202, as described in U.S. Provisional Patent Application No. 61/935,113, filed Feb. 3, 2014, which is hereby incorporated by reference in its entirety. Generally, any number of electrical conductors, optical pathways, and/or combinations thereof can extend along the length of the flexible elongate member 202 between the connector 210 and the component 208, embedded in the core or not. In some instances, between one and ten electrical conductors and/or optical pathways extend along the length of the flexible elongate member 202 between the connector 210 and the component 208. The number of communication pathways and the number of electrical conductors and optical pathways extending along the length of the flexible elongate member 202 is determined by the desired functionality of the component 208 and the corresponding elements that define component 208 to provide such functionality.

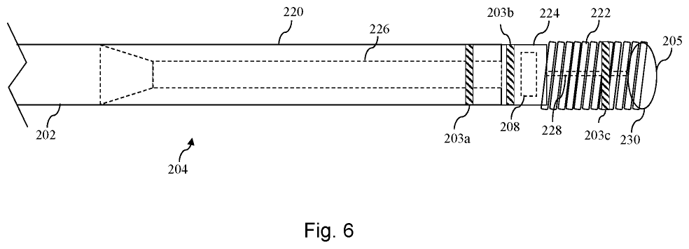

Referring now to FIG. 6, shown therein are examples of implementations of the radiopaque marker 203 on the intravascular device 200 according to the present disclosure. In particular, FIG. 6 provides a diagrammatic, schematic side view of the distal portion 204 of the intravascular device 200. As shown, the distal portion 204 includes a proximal flexible element 220 and a distal flexible element 222 on each side of a housing 224 containing component 208. A core member 226 extends through the proximal flexible element 220. Similarly, a core member 228 extends through the distal flexible element 222. In some implementations, the core members 226 and 228 are an integral component (i.e., the core member 226 extends through the housing 224 and to define core member 228). Generally, the core members 226, 228 are sized, shaped, and/or formed out of particular material(s) to create a desired mechanical performance for the distal portion 204 of the intravascular device 200. In that regard, in some instances the core member 228 is coupled to a shaping ribbon. For example, in some particular implementations the core member 228 is coupled to a shaping ribbon utilizing a multi-flat transition as described in U.S. Provisional Patent Application No. 62/027,556, filed Jul. 22, 2014, which is hereby incorporated by reference in its entirety.

The proximal and distal flexible elements 220, 222 can be any suitable flexible element, including coils, polymer tubes, and/or coil-embedded polymer tubes. In the illustrated embodiment the proximal flexible element 220 is a coil-embedded polymer tube and the distal flexible element 222 is a coil. As discussed in greater detail below, the proximal and/or distal flexible elements 220, 222 are at least partially filled with one or more flexible adhesives to improve the mechanical performance and durability of the intravascular device 200. In that regard, in some instances adhesives with varying degrees of durometer are utilized to provide a desired transition in bending stiffness along the length of the intravascular device 200. A solder ball 230 or other suitable element is secured to the distal end of the distal flexible element 222. As shown, the solder ball 230 defines the distal tip 205 of the intravascular device 200 with an atraumatic tip suitable for advancement through patient vessels, such as vasculature. In some embodiments, a flow sensor is positioned at the distal tip 205 instead of the solder ball 230.

FIG. 6 illustrates an embodiment in which the radiopaque marker 203a is coupled to the proximal flexible element 220, the radiopaque marker 203b is coupled to the housing 224, and the radiopaque marker 203c is coupled to the distal flexible element 222. The illustrated arrangement is exemplary and the radiopaque marker 203 can be coupled to any component of the intravascular device. The radiopaque marker can have any suitable length, extending along a majority of the length of the intravascular device 200, including along the flexible elongate member 202 (e.g., along all, a majority, and/or a portion of the distance between the connector 210 and the proximal flexible element 220 in some instances), between the proximal flexible element 220 and the distal flexible element 222, and/or the distal flexible element 222. It is understood that the radiopaque marker 203 may be applied to any combination of areas and/or components of the intravascular device 200, including in the axial (along the length of the device) and/or circumferential (around the circumference of the device) directions. For example, the radiopaque marker 203 may be applied partially around the circumference of the device (e.g., half, ¼, ¾, or other suitable amount) in certain areas.

The distal portion 204 of the intravascular device 200—as well as the proximal portion 206 and the flexible elongate member 202—may be formed using any suitable approach so long as a portion of the intravascular device includes the radiopaque marker 203 in accordance with the present disclosure. Accordingly, in some implementations the intravascular device 200 includes features similar to the distal, intermediate, and/or proximal sections described in one or more of U.S. Pat. Nos. 5,125,137, 5,873,835, 6,106,476, 6,551,250, U.S. patent application Ser. No. 13/931,052, filed Jun. 28, 2013, U.S. patent application Ser. No. 14/135,117, filed Dec. 19, 2013, U.S. patent application Ser. No. 14/137,364, filed Dec. 20, 2013, U.S. patent application Ser. No. 14/139,543, filed Dec. 23, 2013, U.S. patent application Ser. No. 14/143,304, filed Dec. 30, 2013, and U.S. Provisional Patent Application No. 61/935,113, filed Feb. 3, 2014, each of which is hereby incorporated by reference in its entirety.

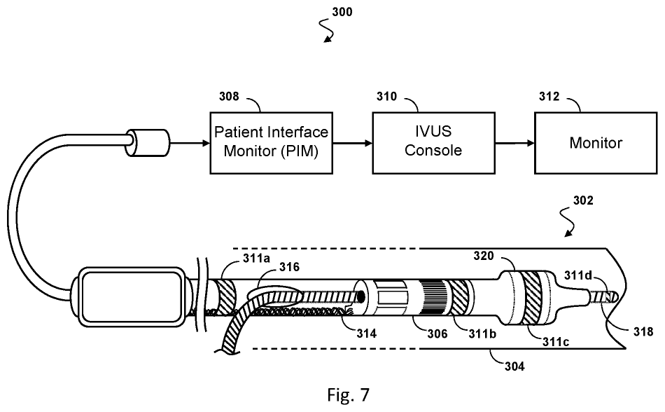

FIG. 7 is a diagrammatic schematic view of an ultrasound imaging system 300 according to an embodiment of the present disclosure. The distal-most end of the elongate member 302 includes a scanner assembly 306 with an array of ultrasound transducers and associated control circuitry. When the scanner assembly 306 is positioned near the area to be imaged, the ultrasound transducers are activated and ultrasonic energy is produced. A portion of the ultrasonic energy is reflected by the vessel 304 and the surrounding anatomy and received by the transducers. Corresponding echo information is passed along through a patient interface module (PIM) 308 to an IVUS console 310, which renders the information as an image for display on a monitor 312.

The imaging system 300 may use any of a variety of ultrasonic imaging technologies. Accordingly, in some embodiments of the present disclosure, the IVUS imaging system 300 is a solid-state IVUS imaging system incorporating an array of piezoelectric transducers fabricated from lead-zirconate-titanate (PZT) ceramic. In some embodiments, the system 300 incorporates capacitive micromachined ultrasonic transducers (CMUTs), or piezoelectric micromachined ultrasound transducers (PMUTs).

In some embodiments, the IVUS system 300 includes some features similar to traditional solid-state IVUS system, such as the EagleEye® catheter available from Volcano Corporation and those disclosed in U.S. Pat. No. 7,846,101 hereby incorporated by reference in its entirety. For example, the elongate member 302 includes the ultrasound scanner assembly 306 at a distal end of the member 302, which is coupled to the PIM 308 and the IVUS console 310 by a cable 314 extending along the longitudinal body of the member 302. The cable 314 caries control signals, echo data, and power between the scanner assembly 306 and the remainder of the IVUS system 300. In some instances, the scanner assembly 306 is transitioned from a flat configuration to a rolled or more cylindrical configuration. For example, in some embodiments, techniques are utilized as disclosed in one or more of U.S. Pat. No. 6,776,763, titled “ULTRASONIC TRANSDUCER ARRAY AND METHOD OF MANUFACTURING THE SAME” and U.S. Pat. No. 7,226,417, titled “HIGH RESOLUTION INTRAVASCULAR ULTRASOUND TRANSDUCER ASSEMBLY HAVING A FLEXIBLE SUBSTRATE,” each of which is hereby incorporated by reference in its entirety.

In an embodiment, the elongate member 302 further includes a guide wire exit port 316. The guide wire exit port 316 allows a guide wire 318 to be inserted towards the distal end in order to direct the member 302 through a vascular structure (i.e., a vessel) 304. Accordingly, in some instances the IVUS device is a rapid-exchange catheter. In an embodiment, the elongate member 302 also includes an inflatable balloon portion 320 near the distal tip. The balloon portion 320 is open to a lumen that travels along the length of the IVUS device and ends in an inflation port (not shown). The balloon 320 may be selectively inflated and deflated via the inflation port. In some embodiments, the catheter does not include a balloon 320. Rather, a flexible, polymeric tip member is provided distal of the scanner assembly. The tip member can be a leading component of the catheter while the catheter traverses through vasculature.

The PIM 308 facilitates communication of signals between the IVUS console 310 and the elongate member 302 to control the operation of the scanner assembly 306. This includes generating control signals to configure the scanner, generating signals to trigger the transmitter circuits, and/or forwarding echo signals captured by the scanner assembly 306 to the IVUS console 310. With regard to the echo signals, the PIM 308 forwards the received signals and, in some embodiments, performs preliminary signal processing prior to transmitting the signals to the console 310. In examples of such embodiments, the PIM 308 performs amplification, filtering, and/or aggregating of the data. In an embodiment, the PIM 308 also supplies high- and low-voltage DC power to support operation of the circuitry within the scanner assembly 306.

The IVUS console 310 receives the echo data from the scanner assembly 306 by way of the PIM 308 and processes the data to create an image of the tissue surrounding the scanner assembly 306. The console 310 may also display the image on the monitor 312.

The ultrasound imaging system 300 may be utilized in a variety of applications and can be used to image vessels and structures within a living body. Vessel 304 represents fluid filled or surrounded structures, both natural and man-made, within a living body that may be imaged and can include for example, but without limitation, structures such as: organs including the liver, heart, kidneys, as well as valves within the blood or other systems of the body. In addition to imaging natural structures, the images may also include imaging man-made structures such as, but without limitation, heart valves, stents, shunts, filters and other devices positioned within the body.

In accordance with the present disclosure, a radiopaque marker 311 is applied to at least a portion of the elongate member 302. In this regard, the radiopaque marker 311a is coupled to the elongate member 302 proximal of the exit port 316, the radiopaque marker 311b is coupled to the scanner assembly 306, and the radiopaque marker 311c is coupled to the balloon 320. In some embodiments, the radiopaque marker 311d is coupled to the guidewire 318. The locations of the radiopaque markers 311 are exemplary. The radiopaque markers 311 can be provided at any desired location and/or on any desired component of the elongate member 302. It is understood that the radiopaque marker 311 may be applied to any combination of areas and/or components of the elongate member 302, including in the axial (along the length of the device) and/or circumferential (around the circumference of the device) directions. For example, the radiopaque marker 311 may be applied partially around the circumference of the device (e.g., half, ¼, ¾, or other suitable amount) in certain areas.

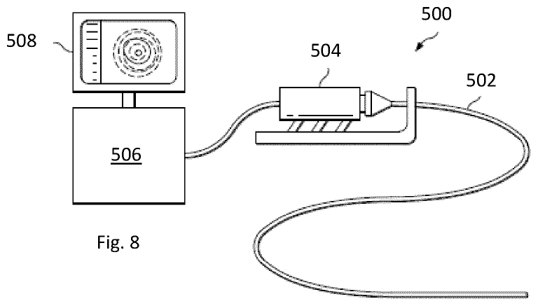

Referring to FIG. 8, shown therein is an IVUS imaging system 500 according to an embodiment of the present disclosure. In some embodiments of the present disclosure, the IVUS imaging system 500 is a lead-zirconate-titanate (PZT), capacitive micromachined ultrasonic transducer (CMUT), or piezoelectric micro-machined ultrasound transducer (PMUT) rotational IVUS imaging system. In that regard, the main components of the rotational IVUS imaging system are the rotational IVUS catheter 502, a catheter compatible patient interface module (PIM) 504, an IVUS console or processing system 506, and a monitor 508 to display the IVUS images generated by the IVUS console 506.

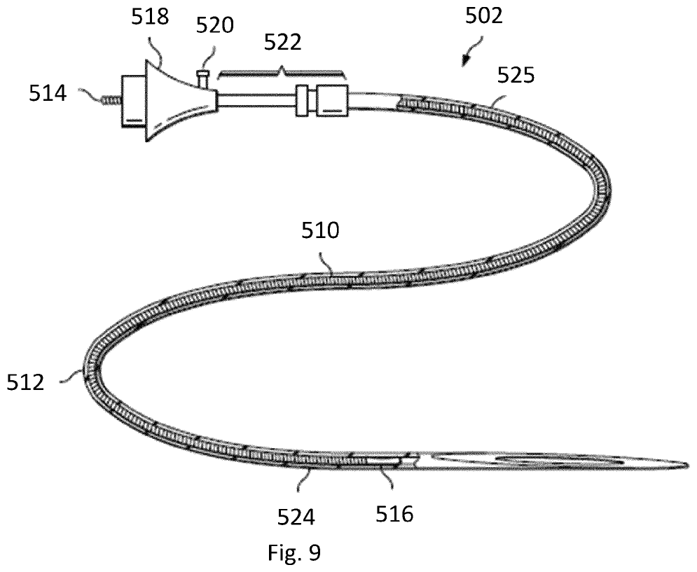

Referring now to FIG. 9, shown therein is a diagrammatic, partial cutaway perspective view of the catheter 502 according to an embodiment of the present disclosure. In that regard, FIG. 9 shows additional detail regarding the construction of the rotational IVUS catheter 502. In many respects, this catheter is similar to traditional rotational IVUS catheters, such as the Revolution® catheter available from Volcano Corporation and described in U.S. Pat. No. 8,104,479, or those disclosed in U.S. Pat. Nos. 5,243,988 and 5,546,948, each of which is hereby incorporated by reference in its entirety. In that regard, the rotational IVUS catheter 502 includes an imaging core 510 and an outer catheter/sheath assembly 512. The imaging core 510 includes a flexible drive shaft that is terminated at the proximal end by a rotational interface 514 providing electrical and mechanical coupling to the PIM 504 of FIG. 8. The distal end of the flexible drive shaft of the imaging core 510 is coupled to a transducer housing 516 containing the PMUT and associated circuitry. The catheter/sheath assembly 512 includes a hub 518 that supports the rotational interface and provides a bearing surface and a fluid seal between the rotating and non-rotating elements of the catheter assembly. The hub 518 includes a Luer lock flush port 520 through which saline is injected to flush out the air and fill the inner lumen of the sheath with an ultrasound-compatible fluid at the time of use of the catheter. The saline or other similar flush is typically required since ultrasound does not readily propagate through air. Saline also provides a biocompatible lubricant for the rotating driveshaft. The hub 518 is coupled to a telescope 522 that includes nested tubular elements and a sliding fluid seal that permit the catheter/sheath assembly 512 to be lengthened or shortened to facilitate axial movement of the transducer housing within an acoustically transparent window 524 of the distal portion of the catheter 502. In some embodiments, the window 524 is composed of thin-walled plastic tubing fabricated from material(s) that readily conduct ultrasound waves between the transducer and the vessel tissue with minimal attenuation, reflection, or refraction. A proximal shaft 525 of the catheter/sheath assembly 512 bridges the segment between the telescope 522 and the window 524, and is composed of a material or composite that provides a lubricious internal lumen and optimum stiffness, but without the need to conduct ultrasound.

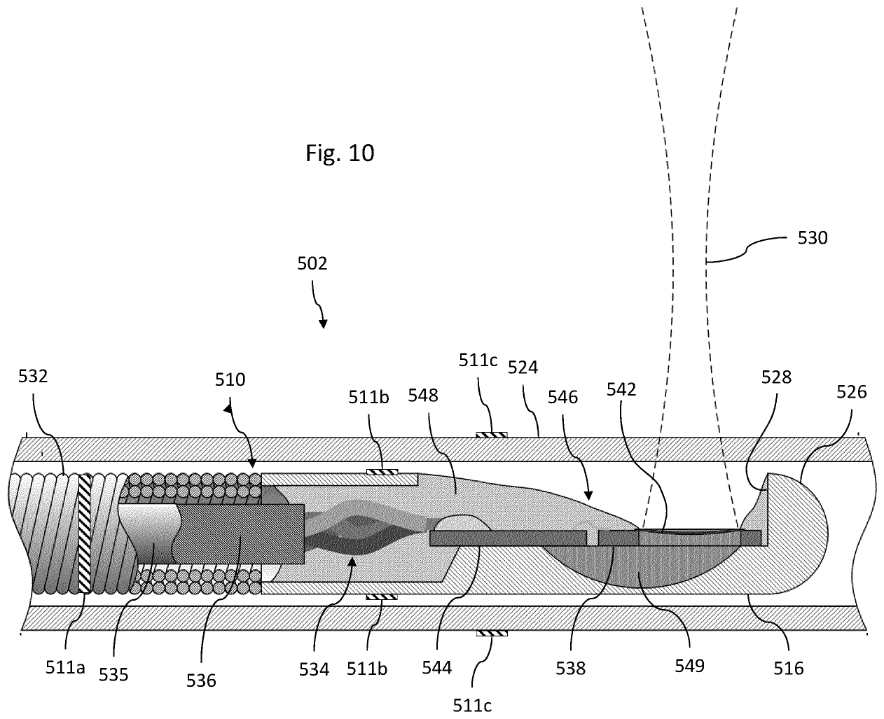

Referring now to FIG. 10, shown therein is a cross-sectional side view of a distal portion of the catheter 502 according to an embodiment of the present disclosure. FIG. 10 shows an expanded view of aspects of the distal portion of the imaging core 510. In this exemplary embodiment, the imaging core 510 is terminated at its distal tip by a housing 516 fabricated from stainless steel and provided with a rounded nose 526 and a cutout 528 for the ultrasound beam 530 to emerge from the housing 516. In some embodiments, the flexible driveshaft 532 of the imaging core 510 is composed of two or more layers of counter wound stainless steel wires, welded, or otherwise secured to the housing 516 such that rotation of the flexible driveshaft also imparts rotation on the housing 516. In the illustrated embodiment, the ASIC 544 and the MEMS 538 components are wire-bonded and glued together to form an ASIC/MEMS hybrid assembly 546, which is mounted to the transducer housing 516 and secured in place with epoxy 548. The leads of the multi-conductor electrical cable 534 with optional shield 536 and jacket 535 are soldered or otherwise electrically coupled directly to the ASIC 144 in this embodiment. The electrical cable 534 extends through an inner lumen of the flexible driveshaft 532 to the proximal end of the imaging core 510 where it is terminated to the electrical connector portion of the rotational interface 114.

When assembled together, as shown in FIG. 10, the MEMS 538 and the ASIC 544 form an ASIC/MEMS hybrid assembly 546 that is mounted within the housing 516, with the ASIC 544 electrically coupled to the MEMS 538 through two or more connections such as wire bonds. In that regard, in some embodiments of the present disclosure the ASIC 544 includes an amplifier, a transmitter, and a protection circuit associated with the MEMS as discussed above. The catheter 502 and/or the MEMS 538 includes an ultrasound transducer 542 configure transmit ultrasound energy and receive ultrasound echoes reflected from the anatomy. The ultrasound beam 530 can be emitted by the ultrasound transducer 542 in any suitable shape. In various embodiments, the transducer 542 is focused or unfocused. In some embodiments, the transducer 542 is spherically focused. In the illustrated embodiment, the connections between the ASIC 544 and MEMS 538 are provided by wire bonds, while in other embodiments, the ASIC 544 is flip-chip mounted to the substrate of the MEMS 538 using anisotropic conductive adhesive or suitable alternative chip-to-chip bonding method. In still other embodiments, both ASIC 544 and MEMS 538 components are attached to a flexible circuit substrate which includes conductive paths to electrically connect the two components. An acoustic backing material 549 can be provided under the transducer 542 to block and/or attenuate propagation of ultrasound energy in undesired directions.

In accordance with the present disclosure, radiopaque marker 511 is applied to at least a portion of the IVUS catheter. In this regard, the radiopaque marker 511a is coupled to the drive cable 532, the radiopaque marker 511b is coupled to the housing 516, and the radiopaque marker 511c is coupled to the acoustically transparent window 524. In some embodiments, the radiopaque marker 511 coupled to the shaft of the catheter 512. The locations of the radiopaque markers 511 are exemplary. The radiopaque markers 511 can be provided at any desired location and/or on any desired component of the catheter 512. However, it is understood that the radiopaque marker 511 may be applied to any combination of areas and/or components of the IVUS catheter, including in the axial (along the length of the device) and/or circumferential (around the circumference of the device) directions. For example, the radiopaque marker 511 may be applied partially around the circumference of the device (e.g., half, ¼, ¾, or other suitable amount) in certain areas.

While the present invention has been described in terms of certain embodiments, those of ordinary skill in the art will recognize that the invention can be practiced with modification within the spirit and scope of the appended claims.

Any spatial references such as, for example, “upper,” “lower,” “above,” “below,” “between,” “bottom,” “vertical,” “horizontal,” “angular,” “upwards,” “downwards,” “side-to-side,” “left-to-right,” “left,” “right,” “right-to-left,” “top-to-bottom,” “bottom-to-top,” “top,” “bottom,” “bottom-up,” “top-down,” etc., are for the purpose of illustration only and do not limit the specific orientation or location of the structure described above.

The following examples are illustrative of the compositions and methods discussed above.

While the present invention has been described in terms of certain embodiments, those of ordinary skill in the art will recognize that the invention can be practiced with modification within the spirit and scope of the appended claims. In particular, it is fully contemplated that the features, components, and/or steps described with respect to one embodiment may be combined with the features, components, and/or steps described with respect to other embodiments of the present disclosure. For the sake of brevity, however, the numerous iterations of these combinations will not be described separately.

The present disclosure has been described relative to certain embodiments. Improvements or modifications that become apparent to persons of ordinary skill in the art only after reading this disclosure are deemed within the spirit and scope of the application. It is understood that several modifications, changes and substitutions are intended in the foregoing disclosure and in some instances some features of the invention will be employed without a corresponding use of other features. Accordingly, it is appropriate that the appended claims be construed broadly and in a manner consistent with the scope of the invention.

What is claimed is:

1. An intraluminal device, comprising:
a catheter or a guidewire comprising a flexible elongate member configured to be positioned within a body lumen of a patient; and
a radiopaque marker coupled to the flexible elongate member, wherein the radiopaque marker is an electromagnetic-radiation-cured product of a mixture comprising:
a radiopaque material;
an electromagnetic-radiation-curable adhesive; and
a photoinitiator.

2. The intraluminal device of claim 1, wherein the electromagnetic-radiation-curable adhesive comprises at least one of an acrylated urethane or an acrylate.

3. The intraluminal device of claim 1, wherein the electromagnetic-radiation-curable adhesive comprises at least one of a monomer or an oligomer.

4. The intraluminal device of claim 1, wherein the radiopaque marker is an ultraviolet (UV)-radiation-cured product.

5. The intraluminal device of claim 1, wherein the mixture comprises a plurality of electromagnetic-radiation-curable adhesives.

6. The intraluminal device of claim 1, wherein the flexible elongate member comprises a surface, and wherein the radiopaque marker conforms to the surface.

7. The intraluminal device of claim 1, wherein the radiopaque marker comprises a band extending around a perimeter of the flexible elongate member.

8. The intraluminal device of claim 1, further comprising:
a sensor disposed at a distal portion of the flexible elongate member and configured to obtain medical data associated with the body lumen while the flexible elongate member is positioned within the body lumen.

9. The intraluminal device of claim 8, wherein the sensor comprises at least one of an ultrasound transducer, a pressure sensor, a flow sensor, or a temperature sensor.

10. The intraluminal device of claim 1, wherein the radiopaque material is between approximately 30% and approximately 90% of a weight of the mixture.

11. A medical device, comprising:
a surface; and
a radiopaque marker coupled to the surface, wherein the radiopaque marker is an electromagnetic-radiation-cured product of a mixture comprising:
a radiopaque material;
an electromagnetic-radiation-curable adhesive; and
a photoinitiator.

12. An intraluminal sensing device, comprising:
a catheter or a guidewire comprising a flexible elongate member configured to be positioned within a body lumen of a patient;
a sensor disposed at a distal portion of the flexible elongate member and configured to obtain medical data associated with the body lumen while the flexible elongate member is positioned within the body lumen, wherein the sensor comprises at least one of an ultrasound transducer, a pressure sensor, a flow sensor, or a temperature sensor; and
a radiopaque marker coupled to the flexible elongate member, wherein the radiopaque marker is an ultraviolet (UV) radiation-cured product of a mixture comprising:
a radiopaque material;
an electromagnetic-radiation-curable adhesive; and
a photoinitiator,
wherein a solution of the mixture is applied directly to the flexible elongate member and UV radiation-cured to form the radiopaque marker,
wherein the radiopaque marker comprises a band extending around a perimeter of the flexible elongate member.
